(19) United States
(12) Patent Application Publication
(10) Pub. No.: US 2006/0217749 A1
(43) Pub. Date: Sep. 28, 2006

(54) Reduced closure force ligating clip

(75) Inventors: Donald F. Wilson JR., Raleigh, NC (US); Steven J. Owens, Loveland, OH (US)

Correspondence Address: BAKER & HOSTETLER LLP, WASHINGTON SQUARE, SUITE 1100, 1050 CONNECTICUT AVE. N.W., WASHINGTON, DC, 20036-5304, US

(73) Assignee: Pilling Weck Incorporated

(21) Appl. No.: 11/089,048

(22) Filed: Mar. 24, 2005

(51) Int. Cl. A61B 17/08

(52) U.S. Cl. 606/157

(57) ABSTRACT

A polymeric, surgical clip having first and second curved legs with each having a pair of opposing side surfaces joined at their proximal ends by a flexible hinge section and movable from an open position to a closed position for clamping a vessel between curved opposing inner surfaces. The first leg terminates at its distal end in a female locking member, and the second leg member terminates in a male locking member complimentary to the female locking member such that when the first and second leg members are moved from an open position to a closed position about the hinge section the male member is lockingly engaged in the female locking member. The clip has a détente positioned on the outer surface of the first leg between the hinge section and the female locking member which serves to urge the first leg to straighten during closure of the clip to reduce the force required to close the surgical clip.

### TECHNICAL FIELD

The present invention relates to surgical clips, and more particularly to a low closure force ligating clip which requires reduced closure force when being applied by an automatic clip applier. Yet more particularly, the present disclosure relates to an improved surgical ligating clip that is provided with a détente on the outer surface of the hook-side leg which serves to straighten the leg and thereby reduce the force required to close the ligating clip when the hook-side leg is contacted by the lower jaw of an automatic clip applier during closing.

### BACKGROUND ART

Many surgical procedures require vessels or other tissues of the human body to be ligated during the surgical process. For example, many surgical procedures require cutting blood vessels (e.g., veins or arteries), and these blood vessels may require ligation to reduce bleeding. In some instances, a surgeon may wish to ligate the vessel temporarily to reduce blood flow to the surgical site during the surgical procedure. In other instances a surgeon may wish to permanently ligate a vessel. Ligation of vessels or other tissues can be performed by closing the vessel with a ligating clip, or by suturing the vessel with surgical thread. The use of surgical thread for ligation requires complex manipulations of the needle and suture material to form the knots required to secure the vessel. Such complex manipulations are time-consuming and difficult to perform, particularly in endoscopic surgical procedures, which are characterized by limited space and visibility. By contrast, ligating clips are relatively easy and quick to apply. Typically, a clip is applied to the vessel or other tissue by using a dedicated mechanical instrument commonly referred to as a surgical clip applier, ligating clip applier, or hemostatic clip applier. Accordingly, the use of ligating clips in endoscopic as well as open surgical procedures has grown dramatically.

Ligating clips can be classified according to their geometric configuration (e.g., symmetric clips or asymmetric clips), and according to the material from which they are manufactured (e.g., metal clips or polymeric clips). Symmetric clips are generally “U” or “V” shaped and thus are substantially symmetrical about a central, longitudinal axis extending between the legs of the clip. Symmetric clips are usually constructed from metals such as stainless steel, titanium, tantalum, or alloys thereof. By means of a dedicated clip applier, the metal clip is permanently deformed over the vessel. An example of one such clip is disclosed in U.S. Pat. No. 5,509,920 to Phillips et al. An example of a metallic clip applier is disclosed in U.S. Pat. No. 3,326,216 to Wood in which a forceps-type applier having conformal jaws is used to grip and maintain alignment of the clip during deformation. Such appliers may additionally dispense a plurality of clips for sequential application, as disclosed in U.S. Pat. No. 4,509,518 to McGarry et al.

With the advent of high technology diagnostic techniques using computer tomography (CATSCAN) and magnetic resonance imaging (MRI), metallic clips have been found to interfere with the imaging techniques. To overcome such interference limitations, biocompatible polymers have been increasingly used for surgical clips. Unlike metallic clips, which are usually symmetric, polymeric clips are usually asymmetric in design and hence lack an axis of symmetry. Inasmuch as the plastic clip cannot be permanently deformed for secure closure around a vessel or other tissue, latching mechanisms have been incorporated into the clip design to establish closure conditions and to secure against re-opening of the vessel. For example, well known polymeric clips are disclosed in U.S. Pat. No. 4,834,096 to Oh et al. and U.S. Pat. No. 5,062,846 to Oh et al., both of which are assigned to the assignee of the presently disclosed subject matter. These plastic clips generally comprise a pair of curved legs joined at their proximal ends with an integral hinge or heel. The distal ends of the curved legs include interlocking latching members. For example, the distal end of one leg terminates in a lip or hook structure into which the distal end of the other leg securely fits to lock the clip in place.

The distal ends of the clips taught in U.S. Pat. No. 5,062,846 to Oh et al. also include lateral bosses that are engaged by the jaws of a clip applier. A clip applier specifically designed for asymmetric plastic clips is used to close the clip around the tissue to be ligated, and to latch or lock the clip in the closed condition. In operation, the jaws of this clip applier are actuated into compressing contact with the legs of the clip. This causes the legs to pivot inwardly about the hinge, thereby deflecting the hook of the one leg to allow reception therein of the distal end of the other leg. A clip applier designed for use with asymmetric plastic clips in an open (i.e., non-endoscopic) surgical procedure is disclosed in U.S. Pat. No. 5,100,416 to Oh et al., also assigned to the assignee of the presently disclosed subject matter.

In addition to compatibility with sophisticated diagnostic techniques, asymmetric clips have other advantages over symmetric clips. For example, because asymmetric clips are formed from polymeric materials, the mouths of asymmetric clips can generally be opened wider than the mouths of symmetric clips. This allows a surgeon to position the clip about the desired vessel with greater accuracy. In addition, a clip of the type described in the aforementioned U.S. Pat. Nos. 4,834,096 and 5,062,846 can be repositioned before locking the clip on the vessel or before removing the clip from the vessel, in a process referred to as “approximating” the clip.

Various types of hemostatic and aneurysm asymmetric clips are used in surgery for ligating blood vessels or other tissues to stop the flow of blood. Such clips have also been used for interrupting or occluding ducts and vessels in particular surgeries such as sterilization procedures.

As known to those skilled in the art, applying the asymmetric ligating clip for occluding the vessel .or other tissue with an automatic clip applier such as the ENDO 5 automatic clip applier available from Pilling Weck Incorporated requires significant additional force to close a clip that has been advanced from the magazine into the jaws of the automatic clip applier. This occurs due to the induced leg flexure of the ligating clip that is created by the spring-loaded clip feeder. The curvature of the ligating clip's two legs allows the axial load of the spring-loaded feeder to cause additional flexure which acts to increase the interference of the female hook locking member with the male locking member so as to require additional force to close the ligating clip and to straighten the two legs of the ligating clip.

Although polymeric surgical ligating clips are well known in the surgical field and improvements have been made to the ligating clips, none have heretofore been designed so as to reduce the force required to close and lock the ligating clip during application with an automatic clip applier such as the ENDO 5 automatic clip applier available from Pilling Weck Incorporated. Therefore, there is believed to be a long-felt need for an improved polymeric surgical ligating clip designed to require a reduced closure force during application by an automatic clip applier. The present disclosure is believed to provide such an improved surgical ligating clip.

### SUMMARY

In accordance with the present disclosure, a polymeric surgical clip is provided of the type comprising first and second legs joined at their proximal ends by a flexible hinge section. Each leg has a vessel clamping inner surface, an opposite outer surface, and a pair of opposing side surfaces. The vessel clamping inner surface is in opposition to the vessel clamping inner surface of the other leg. Further, a female locking member is positioned on the distal end of the first leg and a male locking member is positioned on the distal end of the second leg. The female and male locking members are formed such that when the first and second leg members are moved from an open position to a closed position about the hinge section, the male locking member is lockingly engaged in the female locking member so as to removably lock the clip in the closed position.

The surgical clip further comprises a pair of bosses joined to opposite sides of the first leg, between the hinge section and the distal end of the first leg. The preferred embodiment also comprises a second pair of bosses joined to opposite sides of the second leg at the distal end of the second leg. A sharp tissue-penetrating tooth extends from each of the second pair of bosses outwardly towards the distal end of the first leg.

In the preferred embodiment, the clip has a détente located on the outer surface of the first leg and projecting outwardly therefrom at a location between the flexible hinge and the female locking member. The détente serves to urge the lower or first leg to straighten during closure of the surgical clip by the jaws of an automatic clip applier and consequently to facilitate closure and locking of the surgical clip with reduced closure force being applied to the clip by an automatic clip applier. The détente can have an arcuate shape whereby the radius is no greater than the radius of the pair of bosses at the distal end of the first leg adjacent the female locking member.

Further in the preferred embodiment, the inner vessel-clamping surface of the first leg has a concave radius of curvature and the outer surface has a convex radius of curvature between the hinge section and the distal end. In the same embodiment, the inner vessel-clamping surface of the second leg has a convex radius of curvature and the outer surface has a concave radius of curvature between the hinge section and the distal end. At least one of the inner surfaces of the clip comprises a plurality of protrusions extending from the inner surface, for providing improved vessel retention during and following closure of the clip. Preferably, both of the inner surfaces comprise the plurality of protrusions.

The surgical clip disclosed herein is most suitably made of polymeric material and accordingly minimizes interference with high technology diagnostic modalities such as CAT SCAN, MRI and MRS. At the same time, the clip is nearly as small as comparable metal clips while maintaining sufficient strength and possessing a high degree of security in the clip's latching mechanism. The surgical clip of the discovery is further configured to provide a reduced closure force than conventional polymeric surgical clips when being applied by an automatic clip applier.

It is therefore an object of the presently disclosed surgical clip to provide a reduced closure force polymeric surgical clip.

Some of the objects of the subject matter disclosed herein having been stated hereinabove, other objects will become evident as the description proceeds when taken in connection with the accompanying drawings as best described hereinbelow.

### BRIEF DESCRIPTION OF THE DRAWINGS

FIG. 1 is a perspective view of the reduced closure force ligating clip of the present discovery;

FIG. 2 is a side elevation view of the reduced closure force ligating clip showing the détente on the bottom leg thereof;

FIGS. 3A-3E are schematic views of the closing of a conventional asymmetric ligating clip by the jaws of an automatic clip applier;

FIGS. 4A-4E are schematic views of the closing of the reduced closure force ligating clip by the jaws of an automatic clip applier;

FIGS. 5A-5E are schematic views of the closure of a conventional asymmetric surgical ligating clip about a vessel by the jaws of an automatic clip applier;

FIGS. 6A-6E are schematic views of the closure of the jaws of the reduced closure force surgical ligating clip about a vessel by the jaws of an automatic clip applier;

FIG. 7 is a graph of the force required to close a conventional surgical clip and the reduced closure force surgical clip of the discovery by an automatic clip applier (closure about a vessel);

FIG. 7A is a graph showing the force required to close a conventional surgical ligating clip and the force required to close the reduced closure force ligating clip both when the clips are closed without a vessel therebetween and with a vessel being positioned therebetween;

FIG. 8A is a perspective view of a clip applier being inserted into a compartment of a clip cartridge to engage a surgical ligating clip provided in accordance with the present discovery;

FIG. 8B is another perspective view showing the clip applier engaging the surgical ligating clip loaded in one of the compartments of the clip cartridge as shown in FIG. 8A; and

FIG. 8C is another perspective view showing the clip applier extracting the surgical clip from the compartment of the clip cartridge shown in FIG. 8A.

### DETAILED DESCRIPTION

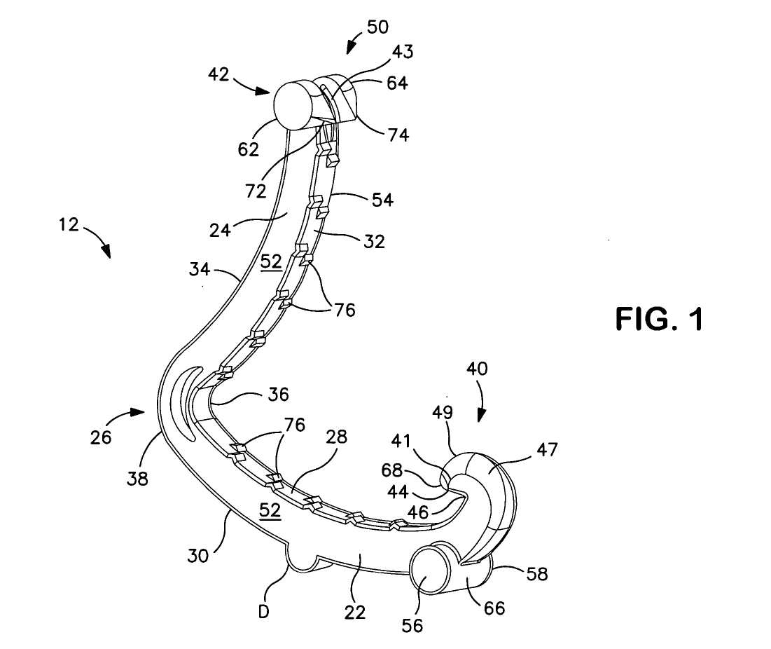

Referring first to FIGS. 1-8 of the drawings, one example is illustrated of an asymmetric surgical clip 12 with a détente D provided on the bottom leg to reduce closure force in accordance with the presently disclosed subject matter. Clip 12 and others of similar design are particularly useful as hemostatic clips that can be latched around a vessel or other type of tissue to ligate the vessel and thereby stop or reduce the flow of fluid through the vessel. Clip 12 can be constructed from any suitable biocompatible material, such as certain metals and polymers. However, the presently disclosed subject matter is particularly suitable for practice with polymeric clips. Thus, clip 12 preferably comprises a one-piece integral polymeric body formed from a suitable strong biocompatible engineering plastic such as the type commonly used for surgical implants. Examples include, but are not limited to, acetyl polyoxymethylene (POM), polyethylene terephthalate (PET), polybutylene terephthalate (PBT), polyoxymethylene, or other thermoplastic materials having similar properties that can be injection-molded, extruded or otherwise processed into like articles.

Now turning to FIG. 1, the body of clip 12 comprises a first or outer leg, generally designated 22, and a second or inner leg, generally designated 24. First and second legs 22 and 24 are joined at their proximal ends by an integral hinge section, generally designated 26. First and second legs 22 and 24 each have a pair of opposing side surfaces 52 and 54. First and second legs 22 and 24 also have complementary arcuate profiles. Thus, first leg 22 has a concave inner surface 28 and a convex outer surface 30, and second leg 24 has a convex inner surface 32 and a concave outer surface 34. Convex inner surface 32 of second leg 24 and concave inner surface 28 of first leg 22 have substantially matching radii of curvature. Detente D is provided on the convex outer surface 30 of first leg 22.

Hinge section 26 has a continuous concave inner surface 36 and a continuous convex outer surface 38. Concave inner surface 36 of hinge section 26 joins concave inner surface 28 of first leg 22 and convex inner surface 32 of second leg 24. Convex outer surface 38 of hinge section 26 joins convex outer surface 30 of first leg 22 and concave outer surface 34 of second leg 24.

First leg 22 terminates in a female locking member 40 at its distal end. Female locking member 40 comprises a resilient inwardly turned hook 41. Second leg 24 terminates in a male locking member 50. Male locking member 50 comprises a pointed tip section 42 at its distal end. Hook 41 is distally curved inwardly toward hinge section 26, and has a transverse beveled surface 44. Beveled surface 44 and concave inner surface 28 define a latching recess 46, which is adapted for conformally engaging tip section 42 of male locking member 50 in the course of compressing clip 12 into a latched or locked position around a vessel or other tissue.

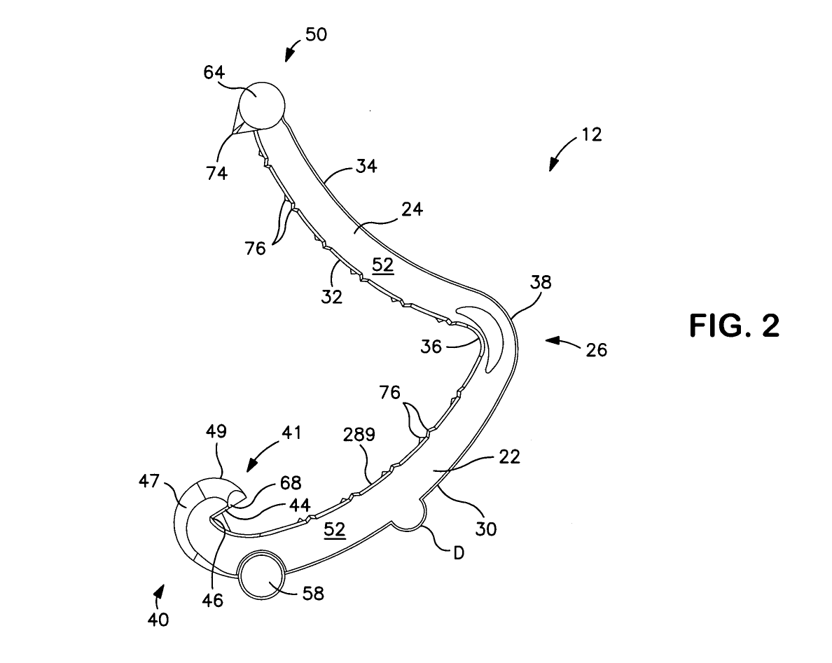

As best shown in FIGS. 1 and 2, the top surface of hook 41 most preferably comprises two convex surfaces 47 that come together to define a sharp crest-like cutting edge 49 to facilitate cutting through connective tissue adjacent a vessel or other tissue during latching of the clip therearound.

As best shown in FIGS. 1 and 2, clip 12 comprises opposing side surfaces 52 and 54. Typically, the body of clip 12 has a constant thickness between side surfaces 52 and 54. Adjacent to the distal end of the first leg 22 and immediately inwardly of hook 41, a pair of cylindrical bosses 56 and 58 are formed coaxially on the opposed side surfaces 52 and 54, respectively, of first leg 22. In the illustrated example of clip 12, a bridge section 66 couples bosses 56 and 58 together. As evident in FIG. 1, bosses 56 and 58 project outwardly beyond convex outer surface 30 of first leg 22.

Referring again to the distal end of second or inner leg 24, another pair of cylindrical bosses 62 and 64 is formed coaxially on the opposed lateral surfaces of inner leg 24 at tip section 42. As evident in FIGS. 1 and 2, bosses 62 and 64 of second leg 24 extend longitudinally forward beyond tip section 42.

Also, as best shown in FIGS. 1 and 2, hook 41 of first leg 22 preferably terminates at a sharp tip 68 with cutting edge 49 extending at least along a portion of the length of the top surface of hook 41. Male locking member 50 of second leg 24 includes a pair of inwardly directed sharp tissue-penetrating teeth 72 and 74, to assist in gripping, stretching and piercing nearby connective tissue, in concert with cutting edge 49 and sharp tip 68 on hook 41.

Both first and second legs 22 and 24 have a plurality of protrusions or teeth 76 extending from their respective inner surfaces 28 and 32. These features are designed to engage the tissue of the vessel being clamped and assist in preventing the vessel from sliding laterally or longitudinally during or following clip closure. It will be noted, however, that other clips equally suitable for use in conjunction with the presently disclosed subject matter may not contain such features.

Clip Closure

In the practice of ligating and cutting a vessel or other tissue, as understood by persons skilled in the art, clip 12 is designed to be compressed into a latched or locked closed position around the vessel through the use of an appropriate clip applicator instrument, such as the ENDO 5 automatic clip applier available from Pilling Weck Incorporated. The clip applicator instrument engages bosses 56, 58, 62 and 64 of clip 12 and pivots bosses 56, 58, 62 and 64 inwardly about hinge section 26. This causes first and second legs 22 and 24 to close around the vessel, with convex inner surface 32 of second leg 24 and complementary concave inner surface 28 of first leg 22 contacting the outer wall of the vessel.

However, before any contact is made between first and second legs 22 and 24, sharp tissue penetrating teeth 72 and 74 on bosses 62 and 64 of second leg 24 start to indent and penetrate any connective tissue surrounding the vessel therebetween and pull the tissue down. Simultaneously, sharp tip 68 and hook 41 on first leg 22, while sliding between teeth 72 and 74, also begin to penetrate the tissue and force the tissue up. Sharp tip 68 and cutting edge 49 on hook 41 enter a groove 43 of pointed tip section 42 on second leg 24, thereby beginning puncturing and cutting of the connective tissue.

As cutting edge 49 and sharp tip 68 of hook 41 continue to move through groove 43 between teeth 72 and 74, shear forces contribute to further puncturing and cutting of the connective tissue surrounding the vessel. If all the tissue is still not cut between the distal portion of second leg 24 and hook 41, it will stretch and become thinner until it is easily punctured by sharp tip 68 and cut by cutting edge 49 of hook 41 as it passes through groove 43 of second leg 24. Once the connective tissue is cut, female and male locking members 40 and 50 are able to lockingly engage without interference.

It should be understood that while cutting edge 49 is a desired feature of the preferred embodiment of clip 12, other embodiments of clip 12 that do not include cutting edge 49 are intended also to be within the scope of the present discovery. Thus, clip 12 may or may not include cutting edge 49 as described above.

Tip section 42 of second leg 24 then begins to contact female locking member 40 at hook 41. Further pivotal movement by the jaws of the applicator instrument longitudinally elongates first leg 22 and deflects hook 41, allowing tip section 42 of male locking member 50 to align with latching recess 46 of female locking member 40. Upon release of the applicator instrument, tip section 42 snaps into and is conformably seated in latching recess 46 of female locking member 40, at which point clip 12 is in its latched and closed position. In the latched condition, tip section 42 is engaged between concave inner surface 28 and beveled surface 44, thereby securely clamping a designated vessel or other tissue between concave inner surface 28 and convex inner surface 32. After clip 12 is secured in its closed position and the vessel is ligated, most likely with two clips 12 on either side of the cutting site, the physician can safely cut the vessel.

Reduced Closure Force Embodiment

To now focus on the novel feature of clip 12 as described and shown herein, applicants note that clip 12 comprises a détente D located on the outer surface 30 of the first leg 22 and that projects outwardly therefrom. Detente D is preferably positioned on outer surface 30 of first leg 22 at a medial location between flexible hinge section 26 and female locking member 40. Detente D thereby serves to urge first leg 22 to straighten during closure of surgical clip 12 and consequently to reduce the closure force required to close and lock surgical clip 12 by an automatic surgical clip applier such as the ENDO 5 available from Pilling Weck Incorporated.

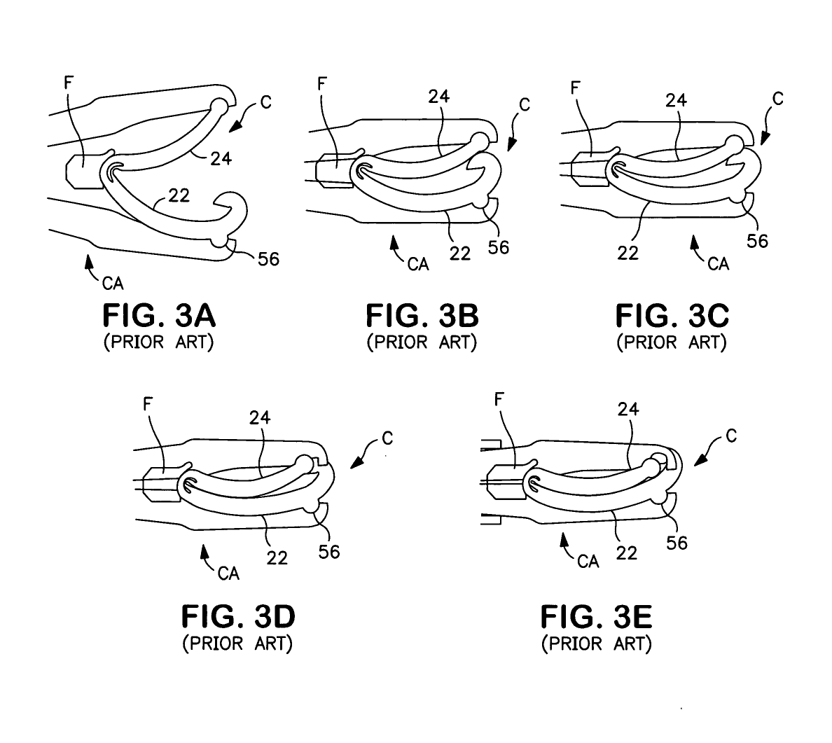

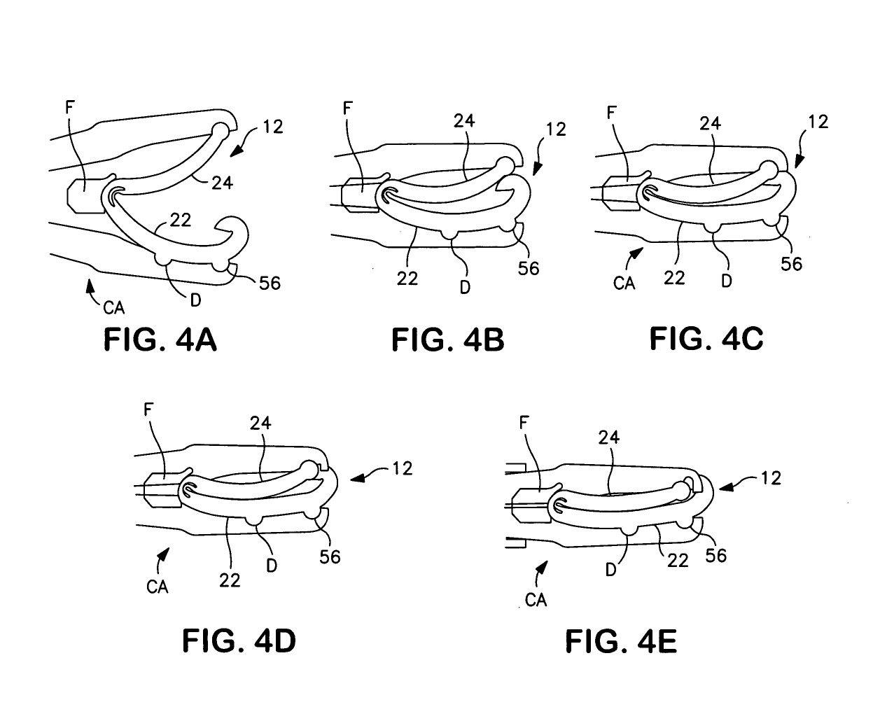

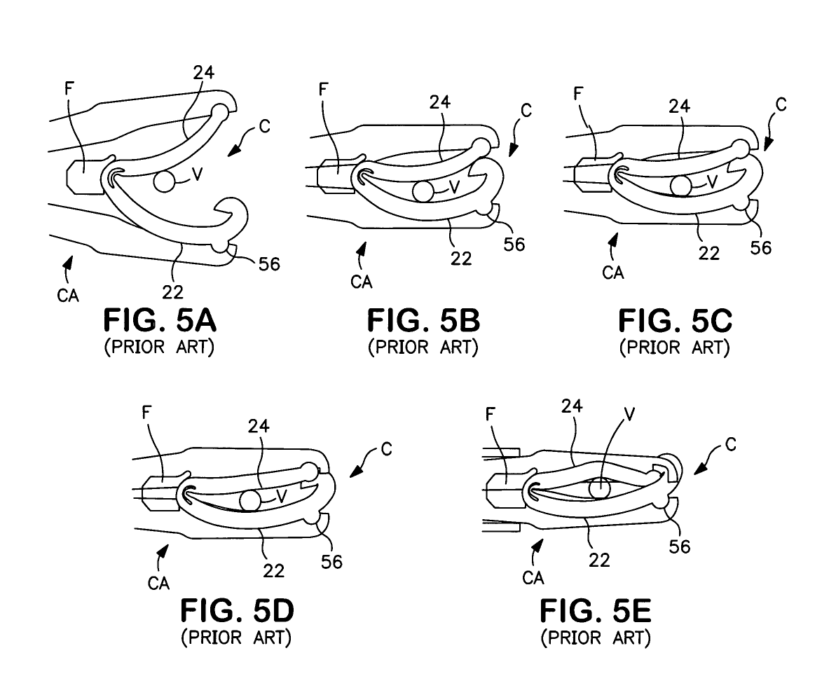

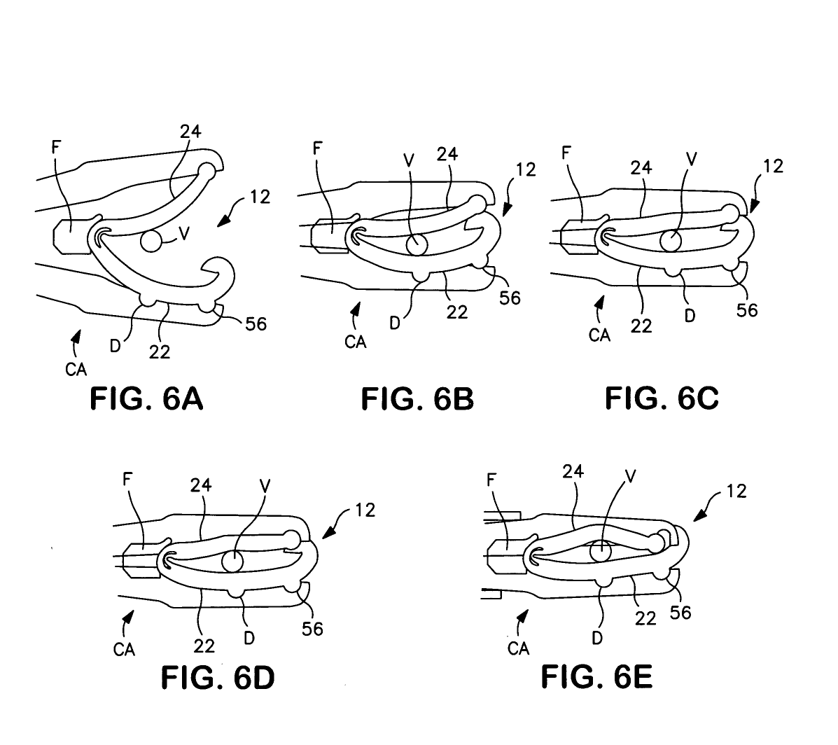

FIGS. 3A to 3E illustrate the closing and locking of a conventional surgical ligating clip C by the jaws of an automatic clip applier CA. FIGS. 4A-4E illustrate the closing and locking of the low closure force clip 12 of the present discovery by the jaws of an automatic clip applier CA. FIGS. 5A-5E indicate the closing and locking of a conventional asymmetric polymeric ligating clip C around a vessel V by the jaws of an automatic clip applier CA. FIGS. 6A-6E are schematic views to illustrate the closing and locking of the low closure force clip 12 of the present discovery about a vessel V by the jaws of an automatic clip applier CA.

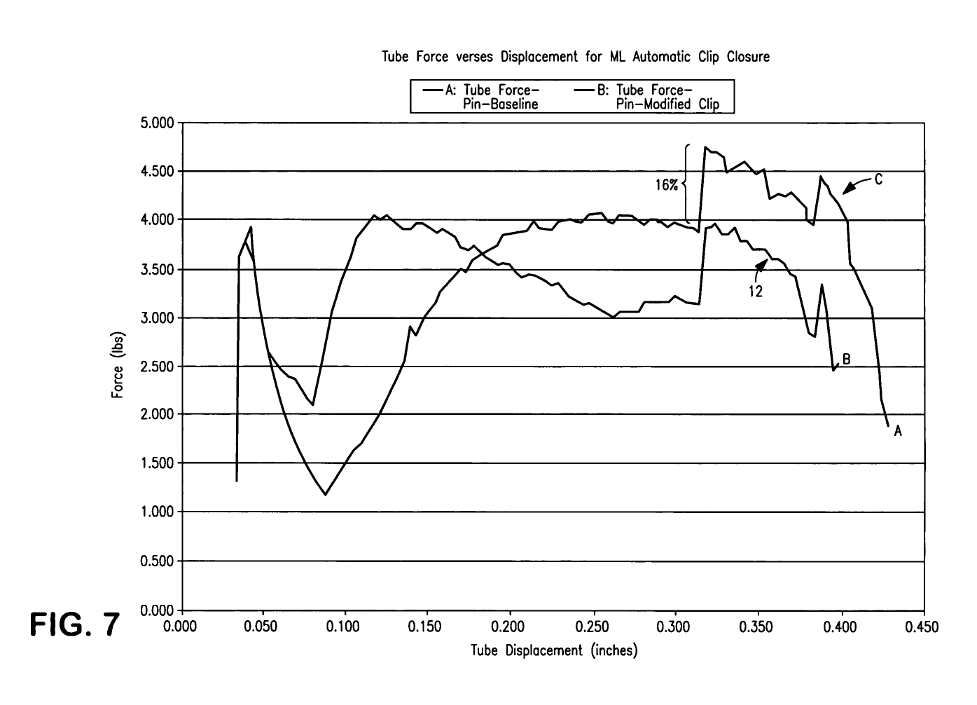

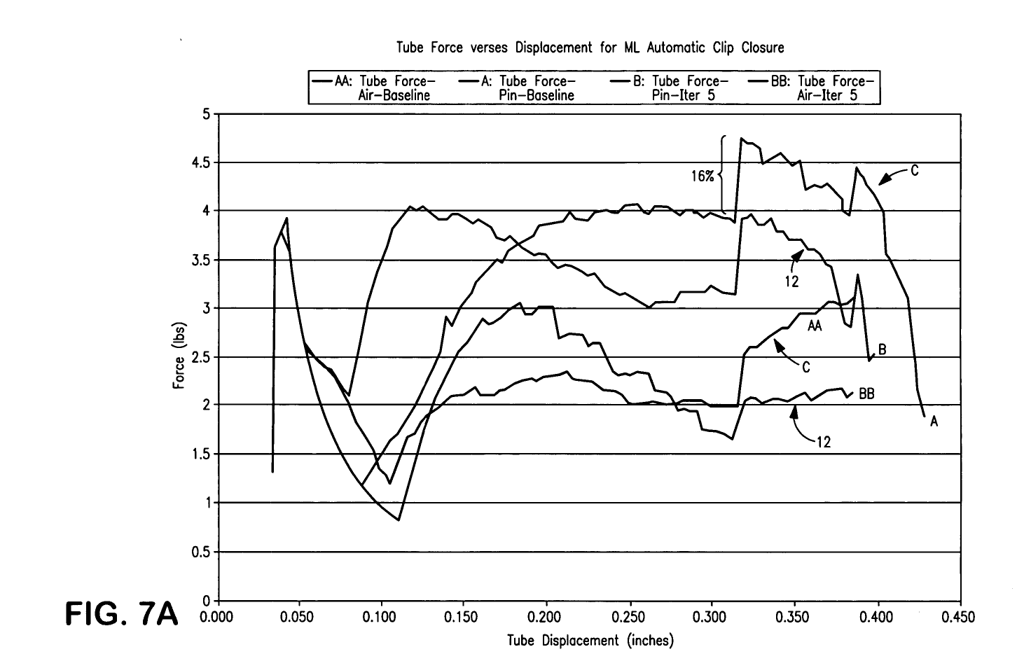

FIG. 7 is a graph which illustrates the force required to close and lock a conventional clip C versus the low closure force clip 12 of the present discovery around a vessel V. As can be seen in FIG. 7, a significantly lower force is required to be applied by the jaws of the automatic clip applier CA in order to close and lock the low closure force clip 12 than is required to close and lock the conventional asymmetric polymeric ligating clip C. FIG. 7A also shows the closing and locking force required for both the conventional asymmetric polymeric ligating clip C and the improved low closure force clip 12 of the present discovery (1) without being clamped around a vessel V and (2) being clamped around a vessel V.

More specifically, détente D on the bottom surface of first leg 22 serves to contact the lower jaw of the automatic clip applier CA during closing of the automatic clip applier jaws. Conventional asymmetric polymeric ligating clips C when utilized in the automatic clip applier require additional force to close and lock the clip due to the induced leg flexure created by the load on spring feeder F at the hinge end of clip C (see FIG. 3). The curvature of legs 22, 24 of clip 12 allows the axial load of the spring feeder F to cause additional flexure. The additional flexure of legs 22, 24 increases the interference of hook 41 of female locking member 40 so as to require additional force to close clip 12 and to straighten legs 22, 24 (see FIG. 4).

Detente D of clip 12 of the present discovery serves to allow leg 22 to better contact the lower jaw of the automatic clip applier CA and to thereby straighten lower first leg 22 and decrease the induced leg flexure which is counteracting the spring feeder force being applied to the back of clip 12 by feeder F of the automatic clip applier CA (see FIG. 4). Preferably, détente D is positioned one-half of the distance between hinge 26 and hook 41. This position applies the straightening force generated by the lower jaw against détente D at the optimum location on first leg 22. The size of détente D is a function of the geometry of the individual automatic clip applier. As illustrated in FIGS. 4 and 6, clip 12 rocks on détente D during initial closing which causes the hook bosses 56, 58, to lift off of the lower jaw. A larger détente D may cause hook bosses 56, 58 to escape the lower jaw which would be undesirable. Thus détente D size needs to be determined integrally with the geometry of the particular automatic clip applier CA being used with clip 12, and preferably it should not protrude out too far from the outer surface of first leg 22 since it may cause first leg bosses 56, 58 to escape the lower jaw of clip applier CA.

As shown in FIG. 7A, the peak closing force of clip 12 is essentially the same when the conventional and the improved asymmetric polymeric ligating clips are closed without a vessel therebetween or “dry fired” (see also FIGS. 3 and 4). The benefit of détente D can be observed in FIGS. 7, 7A when conventional asymmetric polymeric ligating clip C and the improved asymmetric polymeric ligating clip 12 of the present discovery are clamped around a vessel V (see also FIGS. 5 and 6). As seen in FIGS. 7 and 7A, about a 16% reduction in peak closing force is achieved with the improved clip 12 of the present discovery.

Summarily, when the spring feeder F pushes on conventional ligating clip C it tends to bend the lower first leg against the lower jaw of the automatic clip applier CA. The détente D of clip 12 of the present discovery serves to change the pivot point of the lower first leg 22 of clip 12 so that the lower leg 22 tends to deflect outwardly or straighten rather than remain curved toward second leg 24, and thereby reduces the force required to close and lock clip 12 by the jaws of the automatic clip applier CA.

Prior art clips similar to clip 12 are described in detail in the commonly assigned U.S. Pat. Nos. 4,834,096 to Oh et al. and 5,062,846 to Oh et al., the disclosures of which are incorporated herein in their entireties. In addition, the HEM-O-LOK® clip is commercially available from the assignee of the presently disclosed subject matter. These clips are currently available in sizes designated “M”, “ML”, “L” and “XL”. The conventional clip cartridge described hereinbelow can be adapted to accommodate any sizes of HEM-O-LOK® clips commercially available as well as clip 12 of the present discovery.

Clip Cartridge

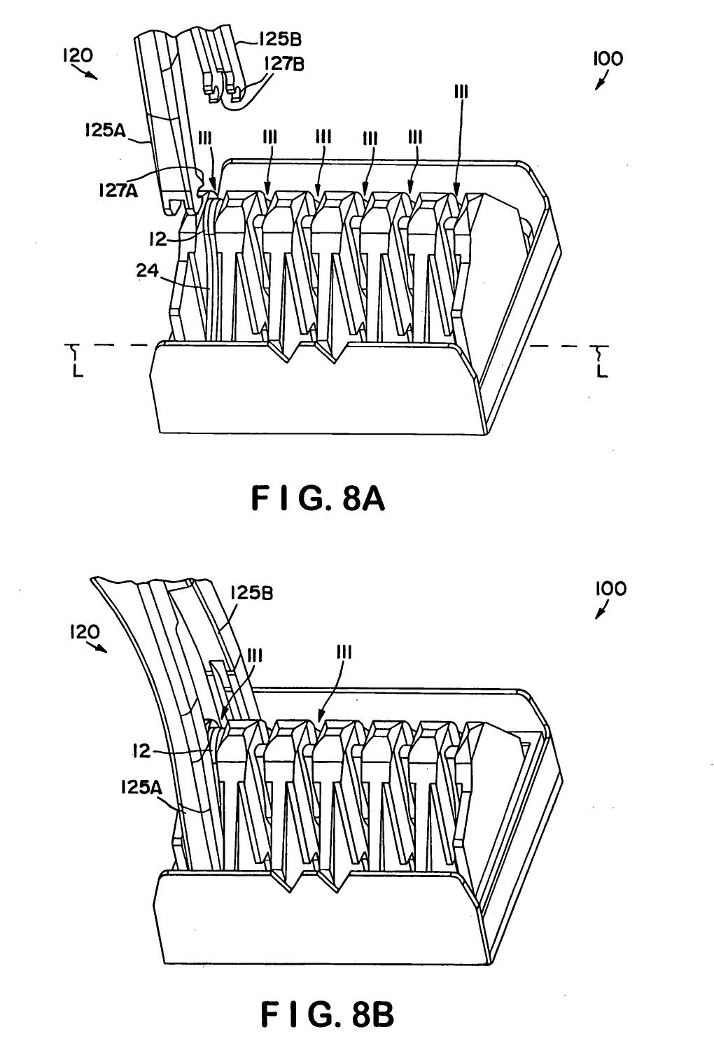

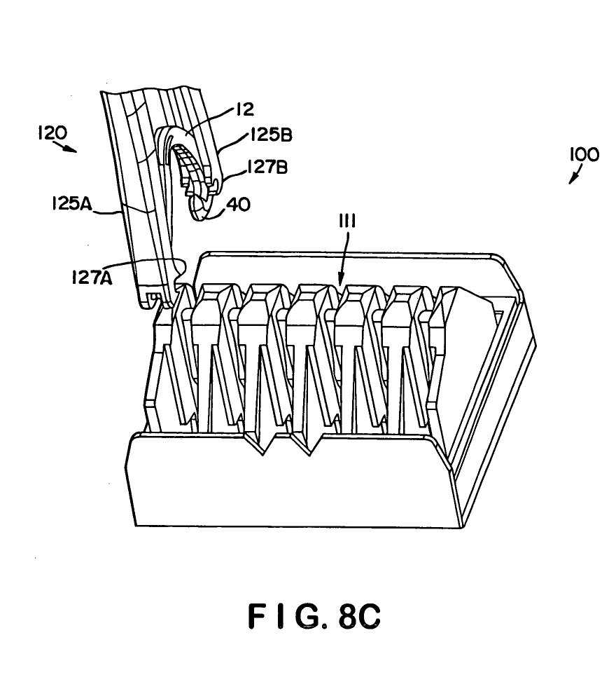

Referring now to FIGS. 8A-8C, a preferred embodiment of a conventional clip cartridge, generally designated 100, is shown for use with clip 12. Clip cartridge 100 preferably is constructed from a single-molded plastic body from which several features are formed. In particular, clip cartridge 100 comprises a plurality of clip retaining chambers or compartments 111 spaced along a longitudinal axis L of clip cartridge 100. Each clip compartment 111 is substantially identical and adapted for storing one clip 12. FIG. 8A illustrates one clip 12 in a stored condition in one of clip compartments 111. It will be understood, however, that preferred embodiments of clip cartridge 100 include several clip compartments 111 for storing several clips 12. For instance, clip cartridge 100 is adapted for storing six clips 12, although other embodiments can be provided that store more or less clips 12. If desired, an adhesive backing (not shown) can be provided on the underside of clip cartridge 100 to facilitate securing clip cartridge 100 to a tray or other supporting component during use.

FIGS. 8A-8C also illustrate the distal end of a representative clip applying instrument for clip 12, generally designated 120, comprising opposing pivotable jaws 125A and 125B. Jaws 125A and 125B have respective jaw recesses 127A and 127B adapted to engage and retain bosses 56, 58, 62 and 64 of clip 12 (see FIGS. 1-2). According to a method provided herein, FIG. 8A illustrates clip-applying instrument 120 in a position over clip 12 prior to inserting clip-applying instrument 120 into a selected clip compartment 111. FIG. 8B illustrates clip-applying instrument 120 being inserted into selected clip compartment 111 to load clip 12 into locking engagement with clip applying instrument 120 (with bosses 56, 58, 62 and 64 retained in jaw recesses 127A and 127B). FIG. 8C illustrates the subsequent step of extracting clip 12 from clip cartridge 100 by removing clip applying instrument 120 with clip 12 loaded therein.

It will be understood that various details of the presently disclosed subject matter can be changed without departing from the scope of the disclosure. Furthermore, the foregoing description is for the purpose of illustration only, and not for the purpose of limitation—the invention being defined by the claims.

What is claimed is:

1. A surgical clip with an integral cutting guide, comprising:
(a) a first leg and a second leg, each of said legs having an inner vessel-clamping surface, an outer surface and a pair of opposing side surfaces flanking said inner and outer surfaces, said inner surfaces being positioned in opposition to each other;
(b) a flexible hinge section integrally disposed between and joining the proximal ends of said first and second legs;
(c) a female locking member positioned on the distal end of said first leg and a male locking member positioned on the distal end of said second leg, said female and male locking members being formed whereby when said first and second leg members are moved from an open position to a closed position about said hinge section, said male locking member is lockingly engaged in said female locking member so as to removably lock said first and second leg members of said surgical clip in said closed position; and
(d) a détente located on said outer surface of said first leg and projecting outwardly therefrom, said détente being positioned on said outer surface of said first leg at a location between said flexible hinge and said female locking member;
whereby said détente serves to urge said first leg to straighten during closure of said surgical clip and consequently to facilitate closure and locking of said surgical clip by a clip applier.

2. The surgical clip of claim 1, wherein said détente is positioned on said outer surface of said first leg at a medial location between said flexible hinge and said female locking member.

3. The surgical clip of claim 2, wherein said détente comprises an arcuate projection.

4. The surgical clip of claim 1, further comprising a pair of bosses joined to opposite sides of said first leg between said hinge section and the distal end of said first leg, and a pair of bosses joined to opposite sides of said second leg at the distal end of said second leg.

5. The surgical clip of claim 4, wherein a portion of said pair of bosses joined to said first leg extends beyond said outer surface of said first leg to form a bridge section.

6. The surgical clip of claim 4, wherein said pair of bosses on said second leg each have a sharp tissue-penetrating tooth extending outwardly therefrom toward said first leg.

7. The surgical clip of claim 1, wherein said inner surface of said first leg has a concave radius of curvature between said hinge section and its distal end, said outer surface of said first leg has a convex radius of curvature between said hinge section and its distal end, said inner surface of said second leg has a convex radius of curvature between said hinge section and its distal end, and said outer surface of said second leg has a concave radius of curvature between said hinge section and its distal end.

8. The surgical clip of claim 1, wherein at least one of said inner surfaces of said clip comprises a plurality of protrusions extending from said inner surface for providing improved vessel retention during and following closure of said clip.

9. The surgical clip of claim 8, wherein both of said inner surfaces of said clip comprise said plurality of protrusions.

10. The surgical clip of claim 1, wherein said hinge section has a continuous concave inner surface and a continuous convex outer surface.

11. The surgical clip of claim 1, wherein said female locking member comprises a resilient inwardly turned hook curved toward said second leg member.

12. The surgical clip of claim 11, wherein said male locking member is complementary to said hook of said female locking member whereby when said first and second leg members are moved from an open position to a closed position about said hinge section, said resilient hook of said female locking member contacts said male locking member and is urged open to receive said male locking member so as to removably lock said first and second leg members of said surgical clip in said closed position.

13. A surgical clip with an integral cutting guide, comprising:
(a) a first leg and a second leg, each of said legs having an inner vessel-clamping surface, an outer surface and a pair of opposing side surfaces flanking said inner and outer surfaces, said inner surfaces being positioned in opposition to each other;
(b) a flexible hinge section integrally disposed between and joining the proximal ends of said first and second legs;
(c) a female locking member positioned on the distal end of said first leg and a male locking member positioned on the distal end of said second leg, said female and male locking members being formed whereby when said first and second leg members are moved from an open position to a closed position about said hinge section, said male locking member is lockingly engaged in said female locking member so as to removably lock said first and second leg members of said surgical clip in said closed position; and
(d) a détente located on said outer surface of said first leg and projecting outwardly therefrom, said détente being positioned on said outer surface of said first leg at a medial location between
said flexible hinge section and said female locking member;
whereby said détente serves to urge said first leg to straighten during closure of said surgical clip and consequently to facilitate closure and locking of said surgical clip by a clip applier.

14. The surgical clip of claim 13, wherein said détente is an arcuate projection.

15. The surgical clip of claim 13, further comprising a pair of bosses joined to opposite sides of said first leg between said hinge section and the distal end of said first leg, and a pair of bosses joined to opposite sides of said second leg at the distal end of said second leg.

16. The surgical clip of 15, wherein a portion of said pair of bosses joined to said first leg extends beyond said outer surface of said first leg to form a bridge section.

17. The surgical clip of claim 15, wherein said pair of bosses on said second leg each have a sharp tissue-penetrating tooth extending outwardly therefrom toward said first leg.

18. The surgical clip of claim 13, wherein said inner surface of said first leg has a concave radius of curvature between said hinge section and its distal end, said outer surface of said first leg has a convex radius of curvature between said hinge section and its distal end, said inner surface of said second leg has a convex radius of curvature between said hinge section and its distal end, and said outer surface of said second leg has a concave radius of curvature between said hinge section and its distal end.

19. The surgical clip of claim 13, wherein at least one of said inner surfaces of said clip comprises a plurality of protrusions extending from said inner surface for providing improved vessel retention during and following closure of said clip.

20. The surgical clip of claim 19, wherein both of said inner surfaces of said clip comprise said plurality of protrusions.

21. The surgical clip of claim 13, wherein said hinge section has a continuous concave inner surface and a continuous convex outer surface.

22. The surgical clip of claim 13, wherein said female locking member comprises a resilient inwardly turned hook curved toward said second leg member.

23. The surgical clip of claim 22, wherein said male locking member is complementary to said hook of said female locking member whereby when said first and second leg members are moved from an open position to a closed position about said hinge section, said resilient hook of said female locking member contacts said male locking member and is urged open to receive said male locking member so as to removably lock said first and second leg members of said surgical clip in said closed position.
